(19) United States
(12) Patent Application Publication
(10) Pub. No.: US 2010/0286722 A1
(43) Pub. Date: Nov. 11, 2010

(54) TEMPORARY VENOUS FILTER SYSTEM

(75) Inventors: Isa Rizk, San Diego, CA (US); John Fulkerson, Rancho Santa Margarita, CA (US); Andrew H. Cragg, Edina, MN (US); George Wallace, Coto de Caza, CA (US)

Correspondence Address: KNOBBE MARTENS OLSON & BEAR LLP, 2040 MAIN STREET, FOURTEENTH FLOOR, IRVINE, CA, 92614, US

(73) Assignee: Intersect Partners, LLC, San Clemente, CA (US)

(21) Appl. No.: 12/463,963

(22) Filed: May 11, 2009

(51) Int. Cl. A61F 2/01

(52) U.S. Cl. 606/200

(57) ABSTRACT

A temporary vascular filter system comprising a catheter and an elongate filter slideably carried near the distal end of said catheter. The vascular filter system can be inserted into a vessel percutaneously with the filter in a narrow-diameter “closed position” and expanded into a large diameter “open position” at the desired intravascular site. After deployment, the proximal portion of the catheter can be secured to the patient at the insertion site. While deployed, the filter component is capable of sliding along a portion of the catheter throughout a range of motion. The filter may have two filter meshes, which may have different degrees of porosity. The temporary vascular filter system can be left in the patient for hours or days and then collapsed into a withdrawal tube for removal from the patient.

### BACKGROUND OF THE INVENTION

1. Field of the Invention

This invention relates generally to filtering systems for placement in a blood vessel and specifically to such systems designed for temporary placement.

2. Description of the Related Art

The body has a well developed-system for forming blood clots (thrombi) that is essential to prevent life-threatening hemorrhages from developing when the vascular system is breached. Unfortunately, the inappropriate activation of the blood clotting system can result in thrombi capable of occluding blood vessels. This can lead to stasis, infarction, and ultimately result in a number of adverse outcomes.

A frequent manifestation of inappropriate clotting is the formation of deep venous thrombi (DVT) in the lower extremities. DVT can cause swelling, pain, and in severe cases, significantly compromise circulation in the extremities. In some cases, all or part of a thrombus can become mobile, forming emboli, a mobile blood clot capable of travelling through the vascular system and doing damage elsewhere. Typically, these emboli travel with the venous blood flow from the lower extremities, through the heart, and into the lungs wherein they can lodge in the pulmonary arteries and cause a condition known as a pulmonary embolism (PE). If the blockage is sufficiently large, the result can be a significant disruption in pulmonary circulation, inadequate oxygenation, and the destruction of lung tissue.

Despite the fact that the incidence of symptomatic PE in the United States is about 650,000 cases annually, the diagnosis and treatment of this common condition is often delayed because of its highly variable presentation. PE can be difficult to treat under the best of conditions, but if left untreated, they can be fatal. It has been estimated that nearly 200,000 Americans die of PE every year.

Emboli that break off from a DVT can sometimes cause additional problems in patients with a patent foramen ovale, or another condition that allows some inappropriate mixing of the arterial and venous blood. In such cases, an embolus can bypass the lungs and directly enter the arterial circulation whereupon it will travel until it becomes lodged in a vessel. The result can be an infarction of the tissue downstream from the clot. While such clots can occur anywhere in the arterial system, they can be especially devastating if they obstruct one of the major arteries of the heart resulting in a myocardial infarction, or if they lodge in one of the arteries supplying the brain and cause a stroke.

Physicians began using pharmacotherapy to prevent and treat DVT and PE in 1938 with the introduction of heparin. Although adequate control can often be achieved using systemic anticoagulation with heparin, enoxaparin, warfarin, or similar medications, 5-20% of patients will experience a second PE even while on anticoagulation. Complications such as hemorrhage and stroke may be as high as 26% with mortality rates ranging from 5-12%. Many patients have conditions that may contraindicate the use of these medications such as pregnancy or the fact that they are about to, or have recently undergone surgical procedures. Nevertheless, anticoagulation remains the mainstay of therapy for patients for DVT and PE.

Surgeons have developed a number of procedures intended to prevent PE. Trousseau proposed inferior vena cava ligation as a possible therapy as early as 1868. In 1934, surgeons began performing femoral vein ligations. However, 10-26% of these patients subsequently developed PE despite having undergone the procedure. Houmans performed the first inferior vena cava (IVC) ligation in 1943. However, this procedure caused a sudden decrease in venous return that resulted in uncompensated cardiac output. Mortality rates as high as 50% ultimately led to the discontinuation of the use of this procedure. Yet another intervention involved the placement of an Adams-DeWeese clip around the IVC which partitioned the lumen of the vessel into four separate channels. While this procedure lowered PE rates to 2-4%, the operative mortality rates ranged from 9-27% and the survivors went on to have IVC thrombosis rates as high as 53%. Surgeons eventually abandoned this procedure once the unacceptably rates of adverse outcomes became apparent.

Venous filters represent another approach to preventing PE and other problems arising from DVT. These are intravascular devices designed so that blood can freely pass through the filter while clots become trapped in the meshwork and are unable to move on to the heart. Such filters are intended to capture potentially fatal emboli at an anatomical location where they pose minimal risk for the patient, i.e., in a large diameter vein where they are unlikely to obstruct blood flow. A variety of geometries have been proposed for venous filters, each having advantages and disadvantages with regard to stopping emboli, facilitating the dissolution of trapped emboli, maximizing blood flow, preventing filter migration, protecting the vessel walls, and maintaining the integrity of the filter itself. Since the vast majority of pulmonary emboli originate from the lower extremities, such filters are usually placed into the IVC. In rare cases there can be an indication to place such filters into the superior vena cava (SVC).

Venous filters and other thrombus trapping devices are generally inserted percutaneously in order to reduce the trauma and risk inherent in more invasive surgical insertion methods. To facilitate insertion, such filters are configured to allow their contraction into a collapsed configuration so that they can be inserted within a narrow tube or catheter. The catheter is normally inserted into a vein and then maneuvered to the desired location under fluoroscopic guidance. Once the catheter is in the desired location, the filter is allowed to expand radially whereupon is held in the desired position via tension against the vessel walls, hooks, or other means of adhesion. Ideally such devices should be designed so that they do not cause any damage to the vessel wall that may result in bleeding or rupture.

The first intra-luminal vena cava filter was the Mobin-Uddin filter. First used in 1967, this device was introduced into the IVC through a venotomy under local anesthesia. It had no appreciable operative mortality rate, and only 3% of patients had recurrent pulmonary emboli. However, the use of this early IVC filter design was ultimately discontinued because of high thrombosis rates as well as venous problems in the lower extremities.

The Kimray-Greenfield filter was first introduced in 1973 and subsequently modified in the 1980s. These filters are constructed of medical-grade stainless steel and featured zig-zag-shaped spokes radiating from a central hub at a 35° angle. The distal ends of the legs are turned upward 180° so as to form hooks for anchoring to the vena cava wall. A variety of such filters are described in U.S. Pat. Nos. 4,688,553 and 4,832,055, the disclosures of which are incorporated herein in their entirety by reference thereto.

The devices described above are commonly thought of as permanent implants. They are expected to remain in the body for more than just a few days or weeks, and are often intended to remain in position for the life of the patient. The use of such filters can lead to numerous possible complications such as the migration of the filter into the heart or lung, the fracture and separation of filter components, the penetration of the IVC by filter components, thrombosis of the vena cava, and an increased incidence of lower extremity deep vein thrombosis. Such filters can also be associated with a high rate of vena cava clot or venous insufficiency symptoms resulting from the inability of the blood to return to the heart in a hemodynamically efficient manner. In such instances, the body attempts to compensate by developing a system of collateral veins. However, such vessels are generally unable to handle the high blood flow required to compensate when the vena cava is substantially obstructed by a filter filled with clots. This can lead to massive swelling of the lower extremities, pain and a marked dilation of lower extremity veins.

In some instances, it may be desirable to implant an IVC filter on a temporary basis. This situation can arise when a patient is preparing to undergo surgery. In such cases, pharmacologic anticoagulation would be strongly contraindicated because of the likelihood of excessive bleeding during the procedure. Another example would be a case wherein a pregnant woman is at risk for thrombosis but possible anticoagulants are contraindicated. In these and other situations, it would be ideal to be able to remove such a filter once the thrombophilic condition has passed or the patient can be started on appropriate medications to treat their condition.

Removing venous filters can be difficult if not outright dangerous. After about two weeks, fibrotic wall reactions lead to endothelialization of the parts of the device in contact with the tunica intima of the lumen. Because the outer edges of the filter become imbedded in vascular tissue, any manipulation after the third week can tear the vessel wall. This can lead to bleeding, thrombus formation, or even dissection of the vessel itself. The latter can result to a life-threatening hemorrhage and necessitate exigent surgical intervention. Because of the high risks involved, the removal of venous filters is generally avoided unless absolutely necessary.

It is essential that an effective temporary filter be capable of performing its intended function, namely entrapping thrombi and decreasing the risk of PE. It is also helpful if such a device is designed so as to facilitate the dissolution of trapped clots in order to maximize blood flow and facilitate removal. In addition, it must be able to remain securely in position, not rotating out of position so that the flow of clots is unimpeded, nor drifting out of its placement site entirely and traveling into the heart. Such a filter would ideally have a small profile during deployment so that it can be placed in difficult to access vasculature, such as that of the brain. Finally, it is important that it be possible to remove the device without damaging the luminal wall of the vessel or the exit point. Unfortunately, a venous filter that combines these ideal characteristics has been heretofore been unavailable.

### SUMMARY OF THE INVENTION

The invention disclosed herein comprises a temporary venous filter system (hereafter “TVFS’). In preferred embodiments, the TVFS comprises an expandable filter, carried by a catheter such that the filter is axially movable along a portion of the catheter throughout a range of motion. At least one of a proximal and a distal stop may be provided, to limit the axial motion of the filter along the catheter.

In preferred embodiments, the filter comprises a tubular body having a substantially cylindrical landing zone which in its radially expanded configuration is dimensioned to seat against the vascular intima of the target vessel lumen. In preferred embodiments, the proximal and distal ends of the filter comprise proximal and distal meshes designed to trap emboli and other debris. In preferred embodiments, said meshes comprise spokes which extend radially outwardly from the centrally positioned catheter to the outer landing zone of the filter thereby providing a barrier across approximately the entire area of the lumen. In many embodiments, the proximal filter has a different mesh size than the distal filter. The filter may be positioned in the vessel such that blood flows first through a more course filter mesh and later passes through a finer filter mesh.

In most embodiments, said filter comprises a proximal collar and distal collar for receiving the catheter. The catheter extends through the proximal collar, throughout the body of the filter and exits through an opening through the distal collar. The filter is able to slide axially along a predetermined portion of the catheter; the movement of the filter being limited by proximal and distal stops on the outer surface of the catheter.

Some embodiments of the TVFS can be inserted into position percutaneously under fluoroscopic guidance. Prior to insertion, the filter can be collapsed into a reduced configuration having only a slightly wider outside diameter than the outside diameter of the catheter. An operator first inserts a delivery guidance device such as a guidewire into a vessel to a point distal to the desired filter placement site. Then the operator guides the catheter and filter over or along the guidewire to the desired location. Once in the proper position, the filter can expand to a diameter roughly equal to that of the vessel lumen. The filter may be restrained by an outer tubular sleeve, which is axially movably carried by the catheter. Proximal retraction of the outer sleeve exposes the filter, which may then self expand to contact the vessel wall. In many embodiments, the guidewire is withdrawn and the proximal catheter manifold is removed. The proximal opening to the guidewire lumen is sealed and the proximal end of the catheter may be secured subcutaneously or at the surface of the skin.

Preferred embodiments of the TVFS can be subsequently removed from the body. To extract the TVFS, the operator exposes the proximal end of the catheter from the body and inserts a capture tube over the catheter. Said capture tube is then advanced distally over the catheter and over the proximal stop to the position of the filter. The filter is then collapsed and drawn into the lumen of the capture tube. Once the collapsed filter is substantially contained within the capture tube, the combination of the capture tube and TVFS can be proximally withdrawn from the body.

The filter of the present invention may be implanted for any of a variety of time periods, depending upon the desired performance. In one implementation of the invention, the filter is implanted prior to heart surgery, and left in position during the surgery and for a post-surgical period of time during which the risk of pulmonary embolism is perceived to remain high. Generally, the filter will remain in position for at least about one or two days, but no more than about 10 days, and often no more than about 5 days. In one implementation of the invention, the filter is positioned pre-surgery, and removed at the time of discharge of the patient from the hospital.

### BRIEF DESCRIPTION OF THE DRAWINGS

FIG. 1 depicts an embodiment of the TVFS 9 with the filter in an open position. The major components of the TVFS 9 comprise a catheter 1 and a filter 17.

FIG. 2a depicts a side view of the filter 17. The filter comprises a filter body 10, proximal 13 and distal 14 filtering meshes, and proximal 11 and distal 12 catheter access collars.

FIG. 2b is a distal end elevational view of the filter 17 rotated 90 degrees from FIG. 2a. This view shows the distal filter mesh 14, the distal catheter access collar 12, and the distal catheter access opening 15.

FIG. 3 depicts the TVFS 9 positioned within the tubular sleeve of a delivery catheter. The filter 17 is restrained by the sleeve in the closed position.

FIG. 4 depicts the removal procedure for the TVFS wherein the capture tube 30 is being advanced distally over the proximal mesh 13 of the filter 17.

### DETAILED DESCRIPTION OF THE PREFERRED EMBODIMENT THE CATHETER PORTION OF THE TVFS

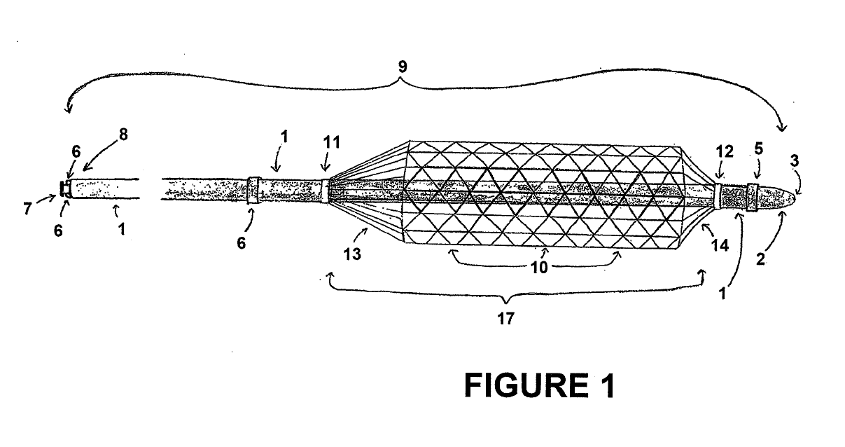

FIG. 1 depicts a temporary venous filter system 9 (TVFS). Preferred embodiments of this invention comprise a catheter 1 and a filter 17. Embodiments of said catheter 1 can comprise a single or multiple lumen extrusion of any of a variety of known biocompatible materials capable of being placed in a blood vessel for an extended period of time, such as PEEK, PEBAX, Nylon, and various densities of polyethylene. Preferred embodiments of said catheter 1 are sufficiently flexible so as to allow an operator to navigate the device through the vascular system during the insertion and withdrawal of the TVFS 9. However, the catheter 1 should be sufficiently rigid so as to provide a stable platform to resist tiling of the filter 17 with respect to the longitudinal axis of the catheter. As the catheter 1 is deployed so that it must hold the filter 17 in position against venous blood flow, preferred embodiments of the catheter 1 can resist folding, bending, or contorting significantly from the pressure of blood flow against the TVFS 9, thus also preventing the filter 17 from moving out of its intended position toward the heart.

The catheter 1 can have any of a variety of cross sectional dimensions. Preferred embodiments of the catheter 1 have a sufficient outside diameter to have a central lumen of sufficient inside diameter to accommodate a guidewire 22. Guidewires having diameters within the range of from about 010 to about 0.035 are presently contemplated, for placing the TVFS 9 in the inferior vena cava. Ideally, the central lumen is sufficiently large so that the catheter 1 can be axially displaceable over the guidewire 22 with minimal resistance. However, preferred embodiments of the catheter 1 also have an outside diameter that is minimized so as to minimize interference with blood flow. Typically, the catheter 1 will have an OD of no more than about 0.091 and often no more than about 0.065 inch.

The length of the catheter 1 will vary, depending upon the desired access site. For example, catheters intended to reach the inferior vena cava from a femoral vein access site will generally have a length within the range of from about 30 cm to about 40 cm. Alternatively, for access via the jugular vein, the axial length will generally be within the range of from about 40 cm to about 60 cm.

In preferred embodiments, the filter component 17 of the TVFS 9 (described in detail below) can have some limited mobility with respect to the catheter 1. Alternatively, either the proximal end or the distal end of the filter component 17 may be axially immovably secured to the catheter. Mobility includes axial mobility along the longitudinal axis of the catheter 1 and/or rotational mobility about the axis of the catheter 1. As will be appreciated from the description below, the filter component 17 is intended for translumenal navigation to a treatment site such as within the inferior vena cava, and radial expansion to bring the filter into direct contact with the vessel wall. The filter 17 in many embodiments remains attached to the catheter 1. The catheter extends proximally through the vasculature to the percutaneous access site. At that site, the catheter 1 may be taped down to the dermal surface or tucked into or below the subcutaneous tissue for the treatment period. During various movement cycles of the patient, and in particular respiration, the effective length of the vasculature between the percutaneous access site and the filter deployment site will change. Since the proximal end of the catheter 1 is relatively immovable fixed, and the length of the catheter 1 is fixed, changes in lung volume and other cyclic movement will have the effect of advancing and retracing the distal end of the catheter 1 with respect to the adjacent vessel wall. By allowing the filter 17 to move axially throughout a range with respect to the catheter 1, the distal end of the catheter 1 can cyclically advance and retract within the vessel in response to respiration, while allowing the filter to remain at its original deployed site thereby enabling respiration to occur without damaging the vascular intima by dragging the deployed filter back and forth within the vessel.

In general, the filter is axially moveable with respect to the catheter throughout a range of at least about 2 mm, often at least about 1 cm, and, in some embodiments, at least about 2 cm. The maximum permitted axial movement is generally no more than about 4 cm.

The catheter 1 may comprise one or both of a proximal stop 6 and a distal stop 5 that are capable of limiting the movement of the filter 17 along the axis of the catheter 1. These proximal and distal stops 5 and 6 may be portions of the catheter 1 wherein the area of the cross section of the catheter 1 through the stops 5 and 6 are greater than the area of the catheter axis openings 15 and 16, thus creating an obstruction that prevents the catheter axis collars 11 and 12 from traveling beyond the proximal stop 6 and distal stop 5. In some embodiments, the proximal and distal stops 6 and 5 can be asymmetrical projections from a portion of the surface of the catheter 1. In some embodiments, the proximal and distal stops 6 and 5 can surround the entirety of the circumference of a portion of the catheter 1, thereby comprising areas of the catheter 1 with a larger diameter than the remainder of the catheter 1.

For example, the proximal stop 6 and/or distal stop 5 may be formed by advancing a short axial length ring formed by a section of tubing concentrically over the catheter shaft to the desired position, where it may be heat shrunk, adhesively bonded or thermally bonded to the catheter shaft. Alternatively, one or both of the proximal collar 11 and distal collar 12 may axially moveable reside in a section of the catheter 1 having a reduced outside diameter. A step such as an annular shoulder separates each of the proximal and distal ends of the recess from the adjacent outside diameter of the catheter. The filter may be axially moveable within the recess, but the proximal and distal shoulders limit axial movement of the collars 11 and 12. Regardless of the proximal and distal stops 5 and 6 exact shape and construction, the proximal and distal stops 5 and 6 form a barrier to limit the movement of the filter 17 and keep the filter component 17 from sliding off of the catheter component 1 of the TVFS 9.

Although the illustrated embodiment provides a proximal collar on the proximal side of the filter and a distal collar on the distal side of the filter, other structures can be utilized to limit axial travel. For example, a single stop such as a collar or two stops may be provided on the catheter shaft within the axial length of the filter, to provide the desired axial range of motion. Alternatively, one stop may be provided within the axial length of the filter and a second stop may be provided on the catheter shaft proximally of the filter or distally of the filter, to entrap either the proximal collar 11 or distal collar 12 within a desired range of motion. The illustrated design, with the proximal and distal stops positioned beyond the ends of the filter, may be the most desirable from a manufacturing perspective.

In some embodiments, the catheter 1 can have at least a second lumen in addition to the guidewire lumen. In most embodiments, the operator can access the second lumen through an access port located at the proximal end 8 of the catheter 1. In some embodiments, the additional lumen can have an exit port on the distal end of the catheter 1 and can be used to introduce contrast dye, thrombolytics, or other medications into the vasculature of the patient.

In some embodiments, an inflation lumen can be provided, to introduce a gas or liquid used to inflate a balloon-like, expandable portion of the catheter 1 capable of applying radial force to the filter 17 thereby pushing the filter 17 from the closed to an open configuration. In most embodiments, such lumens can have an access port at the proximal end 8 of the catheter 1 such as a standard luer connector on the proximal manifold wherein the gas or liquid is introduced, but no distal exit port.

In embodiments of the catheter 1 comprising an expandable portion, this portion can generally be comprised of a hollow flexible bladder or balloon capable of expanding in diameter by elastic expansion or unfolding relative to the remainder of the catheter 1 when a liquid or gas is introduced. In most embodiments, the expandable portion can be located between the proximal and distal stops 6 and 5 near where the body of the filter 10 is positioned during the insertion procedure. In many embodiments, the expandable portion can be made of an elastic material capable of contracting back to its original configuration wherein it is nearly flush with the catheter 1 when the liquid or gas is withdrawn from the catheter 1. This can reduce the cross-section of the catheter 1 post expansion and minimize interference with blood flow when the expandable portion is no longer needed to be inflated after the insertion procedure is complete.

Preferably, however, the temporary filter of the present invention is constructed from a self-expandable metal frame, as is discussed in greater detail below.

The distal openings of any catheter lumens 3, such as a guidewire lumen, can remain open after insertion and positioning at the treatment site. The guidewire may be withdrawn, leaving an open ended guidewire lumen. In many such embodiments, the proximal end 8 of the catheter 1 can have a cap 7, a plug or valve or other means of sealing the proximal access port on guidewire lumen 3 and thereby preventing the escape of blood through the central lumen 3 or other lumens and out the proximal end 8 of the catheter 1. As the flow of blood up the vena cava and against the distal portion of the catheter 1 can be expected to cause some blood to pass into the lumen of the catheter 1 and exit from the proximal opening, the cap 7 or caps can stop this blood from exiting through the proximal portion of the catheter 1.

The passage of blood through an open lumen 3 of the catheter 1 can be prevented by sealing some or all open lumens such as following removal of the guidewire. This can be achieved in a number of ways including the use of an external clamp to simply collapse the guidewire lumen following removal of the guidewire. Alternatively, an internal structure such as a shape memory polymer or alloy can have a biased configuration toward closure of the lumen such as at body temperature, thereby closing the lumen once the guidewire 22 is withdrawn. Other embodiments can seal the central lumen 3 through the use of a second lumen have a proximal access port. When a liquid or gas or push wire is introduced into the access port, it laterally moves a side wall to close the central lumen 3.

The proximal portion of the catheter 1 can have an attachment structure or structures 6 to facilitate the secure attachment of the TVFS 9 to the patient's body. In some embodiments, said attachment structure 6 can comprise one or two or more loops to facilitate suturing. In other embodiments, the attachment may be facilitated through the use of a collar-like attachment or an attachment flange to secure the proximal end 8 of the catheter 1 outside of a blood vessel.

Further features of the catheter will depend upon the configuration of the outer sleeve and/or removal sleeve as are discussed elsewhere herein. For example, in one implementation of the invention, a tubular sleeve is axially moveably carried over the catheter. When the sleeve is in a relatively distal orientation, it surrounds and restrains the filter in a reduced crossing profile configuration. Proximal axial withdrawal of the sleeve over the catheter for a distance of approximately the length of the filter releases the filter which may then radially outwardly expand into contact with the vessel wall. The distal end of the outer sleeve may be left in position over the catheter, such as no more than about 5 cm or 10 cm from the distal end of the catheter. Following the desired treatment period, the outer sleeve may be distally advanced to recapture the filter allowing the assembly to be removed from the patient.

In the foregoing configuration, the catheter may be provided with a proximal manifold which remains attached to the catheter at all times. The outer sleeve may be dimensioned such that it is on the order of 5 or 10 cm shorter than the catheter, so that it may be proximally retracted to release the filter without being impeded by the manifold.

In an alternate configuration, the outer sleeve is intended to be removed from the patient following deployment of the filter. In this implementation, the proximal manifold on the catheter may need to be removed. This may be accomplished either by simply cutting the catheter manifold off using a sharp instrument, or by designing the proximal manifold in a manner that enables disassembly at the clinical site. Alternatively, the outer sleeve may be provided with an axially extending slit, perforated line, or other weakening that allows the outer sleeve to be split and peeled away from the catheter as it is removed over the catheter from the patient.

### The Filter Component of the TVFS

Most embodiments of the filter 17 comprise a self expandable wire or filament frame having several distinct features. These can include but are not limited to one or more filter meshes 13 and 14, a filter body 10, as well as proximal 11 and distal 12 catheter access collars. Said filter 17 can be constructed out of any of a variety of known biocompatible materials suitable for placement within a blood vessel. For example, stainless steel, and shape memory alloys such as Nitinol and Elgiloy, among others, may be used. The filter may be formed from wire stock, such as by forming on a fixture and welding, soldering or otherwise attaching at selected points. Alternatively, at least the body 10 may be formed by laser cutting or otherwise etching from tube stock, as is well understood in the stent arts.

In some embodiments, some of all of the surfaces of the filter 17 can be provided with an active coating such as an antithrombogenic coating. In some embodiments, the outer surface of the filter body 10 can be constructed out of a material or coated by a substance capable of minimizing friction with the endothelium thereby minimizing any stress on the walls of the vessel from the movement of the filter 17.

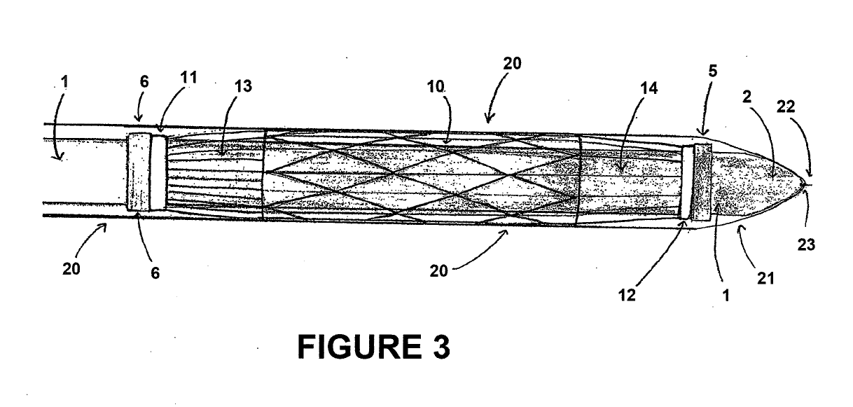

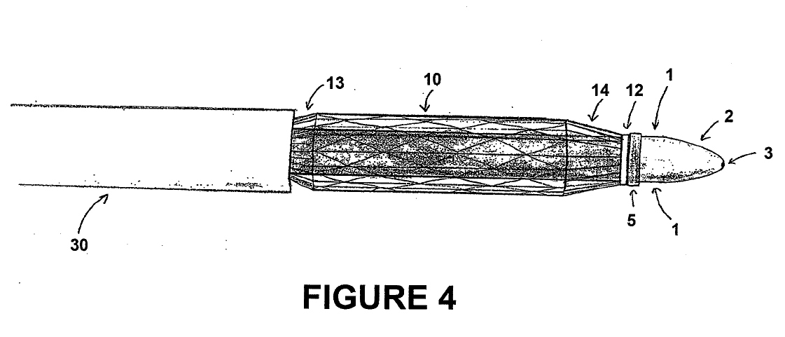

In most embodiments, the filter 17 has several possible configurations including an “open configuration” and a “closed configuration.” The later refers to the configuration of the filter 17 prior to and during insertion (as depicted in FIG. 3) as well as during extraction (as depicted in FIG. 4). While in the closed configuration, the wires of the filter meshes 13 and 14 can be nearly parallel with the axis of the catheter 1 and the filter body 10 is in a compact state so that it is of a relatively small diameter compared to that of the open configuration. In preferred embodiments, the closed configuration has the smallest diameter possible so that the filter 17 can fit within the delivery and extraction tube.

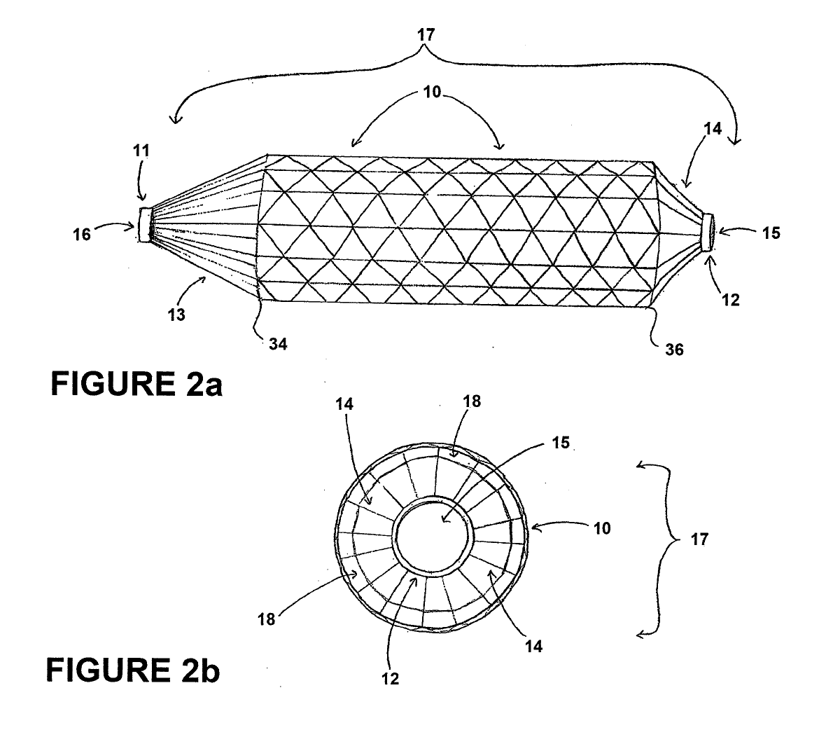

The open configuration refers to the filter 17 when it is deployed for use within a blood vessel (as depicted in FIGS. 1 and 2). In the open configuration, the filter body 10 is expanded so that it is in contact with the wall of the vessel, and all or nearly all of the blood flow passes through the filter meshes 13 and 14.

The catheter access collars 11 and 12 are located at the proximal and distal ends of the filter 17 and encircle the catheter access openings 15 and 16. Most embodiments of said catheter access openings 15 and 16 are circular and are of a slightly greater diameter than the OD of the catheter 1. The catheter 1 extends across the interior of the filter 17 through the proximal opening 16, passes through the interior of the filter body 10, and exits out the distal opening 15. Most embodiments of the filter 17 can axially slide along the catheter 1, however, lateral movement is constricted by the fact the collars 11 and 12 surround the catheter 1 and limit any motion not parallel with the axis of the catheter 1.

In preferred embodiments, the central portion of the filter 17 is comprised of a filter body 10. Said filter body 10 is substantially cylindrical in shape in a bench top expansion, although self expanding embodiments can conform to non-cylindrical anatomies. In most embodiments, the outer surface of said filter body 10 can be configured in the form of a wire lattice. The exact design and configuration of the wire lattice comprising the filter body can vary significantly among various embodiments. In most embodiments, said wires are flexible so that when in a contracted configuration, they can be tightly packed together. When the filter 17 expands into the open configuration, the spaces between the wires within the lattice can grow in size as the internal volume of the filter 17 expands.

The filter body 10 can be manufactured in a number of fully expanded diameters, such as 28 mm. The clinician can select the most appropriate size depending on diameter of the vessel wherein the TVFS 9 is to be deployed. The largest diameter filters 17 can be expected to be deployed in the IVC. However, smaller filters 17 may be used if the TVFS 9 is to be deployed in a smaller vein or artery or a one size fits all configuration capable of accommodating various vessel diameters.

In some embodiments, the filter body 10 comprises a smooth exterior profile for contacting the lumen wall. The outer walls of the filter body 10 can alternatively have a plurality of projections capable of providing traction against the walls of the vena cava thereby maximizing the stability of the deployed filter 17 against blood flow.

In preferred embodiments, the length of the cylindrical filter body 10 is greater than its expanded diameter. This design facilitates the stability of the unit while in position within the body. While in most embodiments, the filter 17 can rotate around the axis of the catheter 1 as well as move along the axis of the catheter 1 to a limited extent, the length of the filter body 10 provides a stable tissue landing zone which can prevent the filter 17 from tumbling or rotating out of position when exposed to blood flow. This maintains filter meshes 13 and 14 on the proximal and distal ends of the filter 17 in a position wherein all or nearly all blood flow passes through the filter meshes 13 and 14.

In general, the landing zone of the filter body 10 extends between a proximal limit 34 and a distal limit 36. In an IVC embodiment of the present invention in which the outside diameter of the filter body 10 in an unconstrained expansion is about 30 mm, the axial length of the landing zone between proximal limit 34 and distal limit 36 is at least about 50 mm, and generally within the range of from about 40 mm to about 80 mm.

In some embodiments, the proximal and distal filter meshes 13 and 14 can comprise a plurality of spoke-like filaments or struts that radiate out from the catheter access collars 11 and 12 to the cylindrical filter body 10. The number of such filaments, and therefore the widths of the spaces between them can determine the relative porosity of the filter meshes 11 and 12. Embodiments with a larger number of filaments generally have a finer filter mesh and can be capable of trapping smaller emboli and other objects. Embodiments comprising fewer filaments generally have a coarser filter mesh.

In some embodiments, the filter meshes 13 and 14 can comprise only the filaments directly connecting the collar and the filter body 10. In some embodiments, the filaments that comprise the filter meshes 13 and 14 can be substantially parallel to the axial direction of the catheter 1 when the filter is in the collapsed position. When the filter 17 is in the open position, the wires of the proximal filter mesh 13 can incline radially outwardly from the proximal catheter access collar 11 to the filter body 10 at an angle within the range of from about 25 to about 70 degrees relative to the axis of the catheter 1 proceeding from the proximal to distal ends.

In the illustrated embodiment, the angle of the wires comprising the distal filter mesh 14 incline in an unconstrained expansion at an angle that is greater (closer to perpendicular) to the longitudinal axis of the catheter (direction of the blood flow) than those of the proximal filter mesh 13. Angles within the range of from about 25 to about 70 are presently contemplated. As will be appreciated, the relative angle of the filter filaments with respect to the longitudinal axis of the implant can affect a variety of characteristics, such as the radial strength of the implant, the effective filter porosity, and also the surface area of the filter surface. In other embodiments, the wires of the distal filter mesh 14 can be symmetrical with those of the proximal filter mesh 13.

FIG. 2b shows a distal end elevational view of the filter 17. In most embodiments, the wires of the proximal 13 and distal 14 filter meshes can appear like spokes connecting the smaller-diameter catheter access collars 11 and 12 to the larger-diameter filter body 10. In some embodiments, the filter meshes 13 and 14 can comprise additional cross-wires 18 connecting adjacent longitudinal filaments. Said cross-wires 18 can be used to add additional structural support to the proximal and distal filter meshes 13 and 14, as well as to further reduce the size of the emboli capable of passing through the meshes 13 and 14. This can be particularly useful near the filter body 10 wherein the distances between the wires of the filter meshes 13 and 14 would be greatest. Either the longitudinal filaments as illustrated in FIG. 2A, or the transverse filaments illustrated in FIG. 2B may be substantially linear in the open configuration, or may be sinusoidal or have any of a variety of wall patterns, depending upon the desired performance characteristics.

In preferred embodiments, the coarser, distal filter mesh 14 can be on the side of the filter that is upstream with respect to the blood flow. Because blood must first pass through the distal filter 14, this can trap larger emboli outside of the filter. The finer, proximal filter mesh 13 can be positioned on the downstream end of the filter 17 and can thereby be capable of trapping smaller emboli and debris capable of passing through the coarser, distal filter mesh 14. This separation of clots by size can help to prevent significant obstructions of a vessel, as can happen if large quantities of emboli are trapped in the same location. In addition, it can help to facilitate the withdrawal of the TVFS 9. If larger clots are trapped within the TVFS 9, these can prevent the filter 17 from collapsing back into a closed position during withdrawal of the TVFS 9.

The effective porosity of the upstream filter may be such that it will let pass particles having a transverse dimension of less than about 4 mm. The effective porosity of the downstream filter may be such that it will let pass particles having a cross-section of less than about 3 mm. The coarseness of either the upstream or downstream filter may be varied considerably, and will be selected depending upon the desired clinical performance. A third filter or a fourth filter may also be included, such as within the interior length of the filter body 10. Alternatively, the filter 17 may be provided with only a single filter element, positioned at the upstream end, the downstream end, or anywhere along the length of the filter body 10.

The orientation of the filter 17 with regard to the catheter 1 can be reversed in some embodiments. The TVFS 9 illustrated in FIG. 1 is configured to be positioned via a proximal insertion point at a supracardiac location while the filter 17 is positioned in an infracardiac location such as the IVC. However, in some embodiments, the TVFS 9 can be inserted into the IVC from an infracardiac location. In such a case, the direction of blood flow will proceed from the proximal end of the catheter 1 toward the distal end of the catheter. Therefore, it would be preferable to have the finer, proximal filter mesh 13 actually facing the distal end 2 of the TVFS 9, and the coarser, distal filter mesh 14 actually facing the proximal end 8 of the catheter 1. The TVFS 9 of the present invention can be easily configured in either orientation such as at the point of manufacture.

### The TVFS and Delivery Tube Prior to Insertion

FIG. 3 depicts the TVFS 9 constrained in a delivery tube 20 prior to insertion. In some embodiments, the device is manufactured and delivered to the operator in this loaded configuration. Alternatively, the filter may be collapsed and loaded within the delivery tube 20 at the clinical site. The delivery tube 20 comprises an elongate flexible tubular body having a proximal end and a distal end with a hollow lumen extending therethrough. Ideally the delivery tube 20 is of as narrow an outside diameter as possible so as to facilitate insertion. However, the lumen must be sufficiently wide so that the TVFS 9 is axially displaceable therefrom. In some embodiments, the surface of the wall defining the central lumen of the delivery tube 20 can be coated with PTFE or other lubricious materials to facilitate the axial displacement of the TVFS 9 from within the delivery tube 20 when the latter is withdrawn.

In preferred embodiments, the delivery tube 20 extends over the length of the catheter 1 to a point distal to the location of the filter 17. In some embodiments, said delivery tube 20 extends beyond the end of the catheter 1. The distal end of the catheter 1 may form or carry a distal cap 21 which covers the distal opening on the delivery tube 20 and provides a smooth, atraumatic surface. In other embodiments, the delivery tube 20 can have a separate tip that covers the distal end 2 of the catheter 1. In such embodiments, the distal end 21 of the delivery tube 20 may be provided with one or more hinged or flexible panels that can be displaced laterally by the TVFS 9 as the delivery tube 20 is being withdrawn over the TVFS 9.

The distal end 21 of the delivery tube 20 is preferably tapered to facilitate entry at the insertion site. Preferred embodiments of the delivery tube 20 have a guidewire access port on the distal end 23.

### Insertion of the TVFS

There are a number of possible insertion techniques for the device herein disclosed. The following description is intended for illustrative purposes only. Persons skilled in the art will recognize that there are numerous possible variations on this technique. The illustration described below should not be construed as limiting the possible techniques whereby the TVFS 9 can be placed into position in a blood vessel or other hollow body structure.

In preferred embodiments, the TVFS 9 can be inserted percutaneously under fluoroscopic guidance using the Seldinger technique or similar procedure for introducing catheters into the vascular system. The insertion site can be in any vein through which the desired filter placement site is accessible and wherein the proximal end of the TVFS 9 can be secured following insertion. Possible supracardiac insertion sites include the jugular, brachiocephalic or subclavian veins. Some embodiments can be inserted at infracardiac locations such as the common iliac or femoral vein. If such embodiments are intended for filter 17 deployment in the IVC, they would likely use the embodiments of the TVFS 9, described above, wherein the finer filter mesh 13 is oriented toward the distal end of the catheter 2.

In preferred embodiments, TVFS 9 is inserted by making a venotomy at the desired insertion site. In most embodiments, the venotomy can be performed using a trocar or similar device. A guidewire 22 is then inserted into the vein. Said guidewire 22 is a narrow wire several meters in length, comprised of a biocompatible material sufficiently rigid so that the operator can direct it down the vascular system, however the guidewire 22 must be sufficiently flexible so that it can be maneuvered through the normal contortions of the vasculature. In preferred embodiments, the length of the guidewire 22 is sufficient for the distal end to reach from the insertion site to the inferior vena cava while still having sufficient length on the proximal side, outside of the patient's body, for the operator to manipulate it with ease. In some embodiments the guidewire 22 can be coated with PTFE or other material to facilitate the ability of the catheter 1 to slide over the guidewire 22. In other embodiments, the guidewire 22 will not have any type of coating.

In most embodiments, the operator directs the guidewire 22 down the superior vena cava, through the right atrium of the heart and into the inferior vena cava. In most embodiments, the guidewire 22 is advanced to a point a few centimeters distal to the desired site of filter 17 placement in the inferior vena cava. Once in the desired position, the operator inserts the proximal end of the guidewire 22 into the distal guidewire access opening of the delivery tube 20 and catheter 3 so that the guidewire 22 is able to pass into the lumen of catheter 1 portion of the TVFS 9 during insertion. The combined delivery tube 20 and TVFS 9 are then threaded down over the guidewire 22 until the end of the delivery tube 20 is positioned distal to the desired insertion location for the filter 17. In many embodiments, the operator can then confirm the position of the filter 17 fluoroscopically, and often with the use of contrast dye. Once the location of the filter 17 has been confirmed, the operator then retracts the delivery tube 20 out from around the TVFS 9, thereby exposing the TVFS 9 in the vena cava. The delivery tube 20 is retracted until it has been removed from the patient and is completely clear of the proximal end 8 of the TVFS 9 and guidewire 22. In some embodiments the guidewire 22 is then retracted. In other embodiments, the guidewire 22 is retracted prior to the removal of the delivery tube 20.

In preferred embodiments, the filter body 10 can expand out to the open configuration wherein it is flush or nearly flush with the endothelium of the vessel. In most embodiments, regardless of the means of expansion, the expansion of the filter body 10 can coincide with an expansion of the open spaces in the wire lattice comprising the filter body 10. Simultaneous with the expansion of the filter body 10 to its final position, the wires of the filter meshes 13 and 14 can move from a configuration wherein they are nearly parallel to the longitudinal axis of catheter 1 to an angle relative to the axis of the catheter 1. During this procedure, the catheter access collars 11 and 12 slide axially along the catheter 1 providing a secure anchoring site for the filter meshes 13 and 14 and the filter body 10.

In some embodiments, the filter body 10 can passively expand into the open position. In such embodiments, the filter body 10 itself can be comprised of a shape memory alloy such as Nitinol that is capable of automatically returning to a specific (biased) shape once deformed out of the preferred shape. In such embodiments, the filter body 10 can be biased to the open configuration. As many such shape memory alloys return to a biased shape at specific temperatures, preferred embodiments of the filter utilizing this technology can be configured to open to their biased shape at physiologic body temperature, typically about 37 degrees Celsius. Most embodiments of such open-biased filters 17 can then be inserted into the delivery tube 20 in the closed position at the point of manufacture. In many such embodiments, the withdrawal of the delivery tube 20 during insertion removes the inward pressure on the filter 17 keeping it in the closed position. This enables the filter 17 to expand to its biased open position in the vessel without the need for the operator to apply mechanical pressure.

In other embodiments, the operator can mechanically expand the filter 17 into the open configuration. In some embodiments, this can be accomplished through the inflation of the expandable portion of the catheter 1. When the operator inflates this segment of the catheter 1, it can mechanically push the exterior surface of the filter body 10 into the open configuration through the application of radial force. In such embodiments, the expandable portion of the catheter 1 can be positioned so that it is directly interior to the filter body 10, thereby facilitating expansion. Following the expansion of the filter 17 into the open position, the operator can then deflate the expandable portion of the catheter 1 back to its original configuration wherein it is substantially flush with the remainder of the catheter 1. Depending upon the strut wall pattern, radial expansion can alternatively be achieved by applying axial compression to the implant.

In some embodiments, a combination of the above techniques described can be used to deploy the filter 17 and maintain it into an open position. In many such embodiments, the filter body 10 can be comprised of a shape memory alloy and be expanded into position mechanically. The shape memory alloy can then serve to maintain the filter 17 in the desired position. In other embodiments, the filter 17 can be constructed so that tension in the wires of the filter mesh or in the wire lattice of the filter body 10 tends to keep the filter 17 in the open position.

In most embodiments, after the operator has placed the filter 17 into the desired position, the operator can then secure the proximal end 8 of the catheter 1 near the insertion site. In some embodiments, the proximal end 8 of the catheter 1 can pass through the lumen of a vessel, and be secured subcutaneously in the tissue near the insertion site. In other embodiments, the proximal end 8 of the catheter 1 can be secured so that it is outside of the patient's body.

### Withdrawal of the TVFS

There are a number of possible techniques whereby the device herein disclosed can be withdrawn from the patient's body. The following description is intended for illustrative purposes only. Persons skilled in the art will recognize that there are numerous possible variations on this technique and the description below should be construed as illustrative and not limiting of all possible techniques.

In preferred embodiments, the TVFS 9 can be withdrawn through the use of a capture tube 30. Said capture tube 30 is a hollow catheter of roughly the same length and width as the insertion tube 20. In some embodiments, the capture tube 30 can be identical to the insertion tube 20. Embodiments of the capture tube 30 can comprise a variety of lengths, most being a meter or longer. Most embodiments are sufficiently long so that the operator can manipulate the proximal end of the capture tube 30 from the insertion site while the distal end of the capture tube 30 can extend over the filter component 17 of the TVFS 9. The capture tube 30 must be sufficiently wide so that the TVFS 9 can fit within the lumen of the capture tube 30. In some embodiments, the lumen of the capture tube 30 can be coated with a lubricious material designed to facilitate the passage of the capture tube over the TVFS 9.

During withdrawal, the operator gains access to the proximal end 8 of the catheter 1 and releases it from its attachment site. In some embodiments, the catheter 1 can be sufficiently long for easy manipulation outside of the patient with little or no risk of the operator inadvertently dropping releasing an unbound TVFS 9 into the vein. In other embodiments, the TVFS 9 can have an attachment whereby the operator can attach an extender to the catheter 1 prior to removal of the TVFS 9 from its proximal attachment site. In some embodiments, this extender can be attached to the catheter 1 using threaded or slip fit couplings, or the catheter attachment sites 6. The extender attachment is preferably accomplished such that the withdrawal tube 30 can be fit over both the extension and the catheter 1 during the withdrawal procedure.

Once the proximal end 8 of the TVFS 9 or its extender is free of its securement site, the operator can slide the proximal end of the TVFS 9 into the distal opening of the capture tube 30. In such embodiments, it will be necessary to gain control of the end of the catheter 1, or the catheter 1 extension out of the proximal portion of the capture tube 30 before inserting the latter into the venotomy site. Once the capture tube 30 is inserted, it is threaded along the catheter 1 until it reaches the proximal filter mesh 13. As most embodiments of the proximal filter mesh 13 comprise wires extending radially outwardly in the distal direction at an acute angle from the axis of the catheter 1, the distal edge of the capture tube 30 can side over them and cause the wires of the filter meshes 13 and 14 to assume a configuration substantially parallel with the catheter 1 (as depicted in FIG. 4). This motion can cause the filter body 10 to contract back into the closed configuration whereupon the capture tube 30 can be advanced over the filter body 10. The operator then continues to advance the capture tube 30 to a location past the filter 17 wherein remaining blood clots trapped beyond the distal filter mesh 14 may enter the capture tube 30 with the blood flow. In many embodiments, the capture tube 30 is advanced to nearly the distal end of the TVFS 9, if not beyond. Once the TVFS 9 is within the capture tube 30, the two components can be withdrawn from the venotomy site together.

In many embodiments, the capture tube 30 can have a seal on the proximal end that minimizes the ability of blood to flow around the TVFS 9 and out through the lumen of the capture tube 30. In some embodiments, the fit between the TVFS 9 and the lumen of the capture tube 30 can be sufficiently tight to largely preclude blood flow up the capture tube 30.

In some embodiments, the operator can inject thrombolytic medications into the guidewire lumen 3 or another lumen of the catheter 1 prior to the extraction procedure. Such medications can exit the catheter 1 at a distal exit point of the lumen and flow back over any emboli trapped on the filter surface. This treatment prior to the extraction procedure may dissolve emboli residing on or within the filter. Obstruction by such emboli can prevent the filter 17 from returning back to the closed position during extraction. Such emboli can also be released during the extraction procedure and subsequently flow up to the heart.

What is claimed is:

1. A vascular filtering system, comprising:
(a) a catheter, having an elongate, flexible body with at least one lumen extending therethrough;
(b) a vascular filtering device carried by, and axially moveable with respect to the catheter; and
(c) at least one stop on the catheter, for limiting the range of axial movement of the filtering device along the catheter.

2. The vascular filtering device of claim 1 wherein said filtering device comprises at least two filtering surfaces.

3. The vascular filtering device of claim 2 wherein the filtering surfaces have different degrees of porosity.

4. The vascular filtering system of claim 1 wherein said filtering device is radially expandable.

5. A vascular filtering device, comprising:
(a) an elongate, tubular body having a proximal end and a distal end, and capable of being placed in a blood vessel;
(b) at least one filtering surface; and
(c) at least one collar configured for slideably receiving a catheter therethrough.

6. The vascular filtering device of claim 5 comprising first and second filtering surfaces having different degrees of porosity.

7. The vascular filtering device of claim 5 wherein the filtering surface comprises a series of wires connecting the collar and the filter body.

8. The vascular filtering device of claim 5 comprising a first collar located on the proximal end and a second collar located on the distal end.

9. The vascular filtering device of claim 5 wherein said filtering device is movable between an open position wherein the diameter of the filter body is relatively large and a closed position wherein the diameter of the filter body is relatively small.

10. The vascular filtering device of claim 5 wherein said filtering device comprises a shape memory alloy.

11. The vascular filtering device of claim 10 wherein the shape memory alloy of said filtering device is biased in an open position.

12. A method of reducing the risk of pulmonary embolism associated with cardiac surgery, comprising the steps of:
translumenally advancing a filter into the inferior vena cava of a patient;
conducting a surgical procedure on the patient; and
withdrawing the filter from the patient following the surgical procedure.

13. A method as in claim 12, wherein the withdrawing the filter step is accomplished within the range of from about one to about 10 days following the translumenally advancing step.

14. A method as in claim 12, further comprising leaving the filter attached to a catheter.

15. A method as in claim 14, comprising permitting the filter to move axially with respect to the catheter throughout a range of motion.

16. A method as in claim 15, wherein the range of motion is at least about 2 mm.

17. An intravascular filter, comprising:
a tubular wire frame, having a proximal end and a distal end;
a proximal filter, on the proximal end of the frame;
a distal filter, on the distal end of the frame; and
an opening on each of the proximal and distal filters for slideably receiving a catheter therethrough;
wherein the proximal filter has a different pore size than the distal filter.
